(19) United States
(12) Patent Application Publication
(10) Pub. No.: US 2009/0076529 A1
(43) Pub. Date: Mar. 19, 2009

(54) Removable support clamp for balloon angioplasty

(76) Inventor: Sastry K. Ganti, Buffalo Grove, IL (US)

Correspondence Address: SASTRY K. GANTI, 1074 CROFTON LANE, BUFFALO GROVE, IL, 60089-4114, US

(21) Appl. No.: 12/011,758

(22) Filed: Jan. 30, 2008

Related U.S. Application Data
(60) Provisional application No. 60/994,152, filed on Sep. 18, 2007.

(51) Int. Cl. A61B 17/08; A61M 25/00

(52) U.S. Cl. 606/151; 604/523

(57) ABSTRACT

An apparatus consists of two or more semi-circular clamshell clamps to surround a lumen externally during balloon angioplasty procedure. When the clamps are closed, they are prevented from opening during the procedure, with the use of a positive stop or a fastener. The procedure facilitates the use of higher than normal pressures within the balloon, which further assists in creating larger passages of the lumen without the fear of rupturing unsupported lumen walls. The procedure also facilitates the use of stronger and larger stents to be implanted internally or externally to the lumen.

### CROSS-REFERENCE TO RELATED APPLICATION

This application claims the benefit of provisional patent application, Ser. No. 60/994152, dated Sep. 18, 2007.

### FEDERALLY SPONSORED RESEARCH

Not applicable

### SEQUENCE LISTING OR PROGRAM

Not Applicable

### BACKGROUND OF THE INVENTION

1. Field of Invention

This invention relates to a temporary, externally placed, removable support clamp around a body lumen, when a specific region of the lumen is subjected to expanding pressure due to balloon angioplasty.

2. Prior Art

When plaque deposits occur on the interior walls of lumens inside a living body; they impede fluid flow. Specifically, atherosclerosis of the walls of arteries of a heart decreases blood supply to the heart and increases the effort of the heart. When the plaque sufficiently narrows the blood passage, or solid particles in the blood block the passage to impede flow to the heart the result may be onset of myocardial infraction. Similar fluid flow blockages can occur for other organs of a body.

A solution to such a problem may be by balloon angioplasty. First, a guide wire is inserted, starting through an incision at a vantage point exterior to the body, through a mutually connected lumen, to the area of narrowed passage and sufficiently beyond it. Then a tube integral with a deflated balloon at its tip, is threaded to the end of the guide wire, precisely to the location of the narrowed passage. The balloon is then inflated, one or more number of times, to controlled pressures and durations. The process radially flattens and redistributes the plaque circumferentially and axially, re-establishing the area required for sufficient fluid flow. The procedure may be followed up with an expandable stent placed around the balloon. Expanding the balloon expands the stent, and anchors it permanently to the interior wall of the lumen. The stent helps in preventing the collapse of the expanded wall of the lumen, and as well may dispense metered medication, which it carries.

If balloon angioplasty cannot be performed, a more involved procedure of ‘bypass surgery’ may be performed. The process involves the steps of removing a piece of healthy lumen having sufficiently large fluid flow passage, from a sacrificial area of the body, reaching the blocked lumen externally through incisions, and attaching the removed lumen from an upstream location of the blockage of the deceased lumen or a different lumen, to the downstream side of the deceased lumen. Sufficient fluid flow is thus re-established by ‘bypassing’ the blocked deceased region to the regions downstream.

### LIMITATIONS OF PRIOR ART

With balloon angioplasty of prior art, the pressure used to flatten the blocking plaque is limited to certain safe levels, so that the lumen walls may not burst, causing loss of fluid under pressure and possible death of the patient.

The limitation of the balloon pressure also limits the size enlargement of the blocked opening that may be achieved, which in turn affects the area available for fluid flow and stent placement. Since the stent further decreases the area for fluid flow, its strength and functionality may be compromised by the meagerness of the radial thickness available in its design and manufacture.

If balloon angioplasty cannot be performed on a patient, due to an excessive extent of blockage or limitation on balloon pressure, the patient is required to go through ‘bypass surgery’. Such surgery puts the patient in a much higher risk of surgical trauma. In case of arteries of the heart, incisions are made externally to reach the blocked arteries and further incisions have to be made at healthier regions upstream and downstream of selected lumens, to suture a healthy lumen segment removed from a different region of the body. To obtain this bypassing lumen, surgery is again required at a healthy region of the body, such as a limb.

### OBJECTS AND ADVANTAGES

Accordingly, the objects and advantages of invention presented in my patent application are directed to a number of patients that are presently turned away from balloon angioplasty and subjected to the riskier and costlier bypass surgery, due to limitation on balloon pressure and strength of unsupported lumen walls.

The new art presented here involves a temporary, externally placed, removable, clamshell type support clamp to the lumen. By using such clamp around the lumen to support it, the balloon can be subjected to higher pressures. Thus flattening the plaque with a larger force is possible than possible with prior art. The larger force will not only permit lumen with higher blockages to be subjected to angioplasty, but also, it will increase the flow area possible when compared to what can be accomplished with prior art.

Further, by using clamshells whose inside diameter is sufficiently larger than the external diameter of the blocked lumen, it will be possible to stretch the lumen permanently to higher inside and outside diameters than what is possible with prior art. The lumen is thus stretched safely to larger diameter than what exists immediately downstream or upstream of it.

This will also allow the placement of a stent of larger inside diameter, greater thickness and hence greater strength, than is possible with prior art.

For the patient, the surgical removal of healthy lumen as a bypass passage will not be necessary, thus avoiding any risks and costs associated with such surgical procedure.

Patients who would normally gain a certain limited enlargement of fluid passage with prior art angioplasty, may gain larger passage when angioplasty is performed with a procedure per invention described here.

### SUMMARY

In accordance with my invention, an apparatus consists of a clamshell type clamp assembly, which is temporarily installed externally around the lumen requiring angioplasty. The apparatus supports the expansion of a lumen wall during balloon angioplasty against radial forces tending to burst the lumen. Following balloon angioplasty as needed, and stent placement if and as needed, the clamp assembly is removed. This described procedure will be possible for some patients who cannot be treated with balloon angioplasty of prior art since the pressure inside the balloon is limited by strength of the unsupported lumen.

### DRAWINGS, FIGURES

FIG. 1 Clamshell type clamp assembly, type A,—open position

FIG. 2 Clamshell type clamp assembly, type B,—open position

FIG. 3 Clamshell type clamp assembly, type A,—closed position

FIG. 4 Clamshell type clamp assembly, type B,—closed position

FIG. 5 Oversized clamp assembly around lumen, type B, closed position

FIG. 6 Cutaway view of overstretched lumen with a stent, type B clamp

FIG. 7 Clamshell type clamp assembly, type C,—closed position

FIG. 8 Section view of clamp assembly, type C, through pin and fastener

FIG. 9 Clamp assembly, type D, with four clamshell halves

FIG. 10 Clamp assembly, type E, with four clamshell halves

FIG. 11 Clamp assembly, type F, with three clamshell halves, assembled

FIG. 12 Clamp assembly, type F, with three clamshell halves, disassembled

FIG. 13 A solid tipped catheter and powered clamshell clamps, wire side view

FIG. 14 A solid tipped catheter and powered clamshell clamps, tip side view

### DRAWINGS, REFERENCE NUMERALS

20 Typical clamshell portion of a clamp, type A
22 Typical extension handle of a clamshell clamp, type A
24 Typical pivot pin at clamshell clamp assembly
26 Typical clamshell portion with extension handle of a clamp, type B
28 U-shaped retainer,—positive stop at type A clamp assembly
30 Wedge block as a retainer,—positive stop at type B clamp assembly
32 Cutaway view of wall of a lumen requiring balloon angioplasty
34 Plaque deposits on lumen's interior wall prior to balloon angioplasty
36 Balloon of catheter (deflated, integral with tube, positioned for angioplasty)
38 Tube of catheter (integral with balloon)
40 Clamshell clamp with handle of type B assembly, with oversized clamshell
42 Radial gap between lumen's outside and clamshell's inside walls
44 End view of lumen laden with plaque, and deflated balloon in position
46 Permanently over-stretched lumen with a stent installed
48 Stent expanded and installed inside over-stretched lumen
50 Clamshell half and handle of type C assembly, with threaded hole feature
52 Clamshell half and handle of type C assembly, with clearance hole feature
54 Clamping fastener at handles of type C clamshell clamp assembly
56 Aligning, anti-rotation pin for Clamshell halves of type C clamp assembly
58 Anti-removal pin-in-groove for clamp's fastener of type C assembly
60 Typical pivot pin for types D and F clamshell clamp assemblies with more than two clamshell halves
62 Clamshell half for type F clamp assembly, with clearance hole feature
64 Clamshell half for type F clamp assembly, with threaded hole feature
66 Non-expandable solid catheter with fluid passages in its tip
68 Typical fluid passage in catheter tip
70 External stent or a similar attachment, over a lumen's wall
72 Clamshell assembly, providing the clamping force against catheter's tip

### DETAILED DESCRIPTION AND OPERATION, PREFERRED EMBODIMENT OF REMOVABLE SUPPORT CLAMP FOR BALLOON ANGIOPLASTY

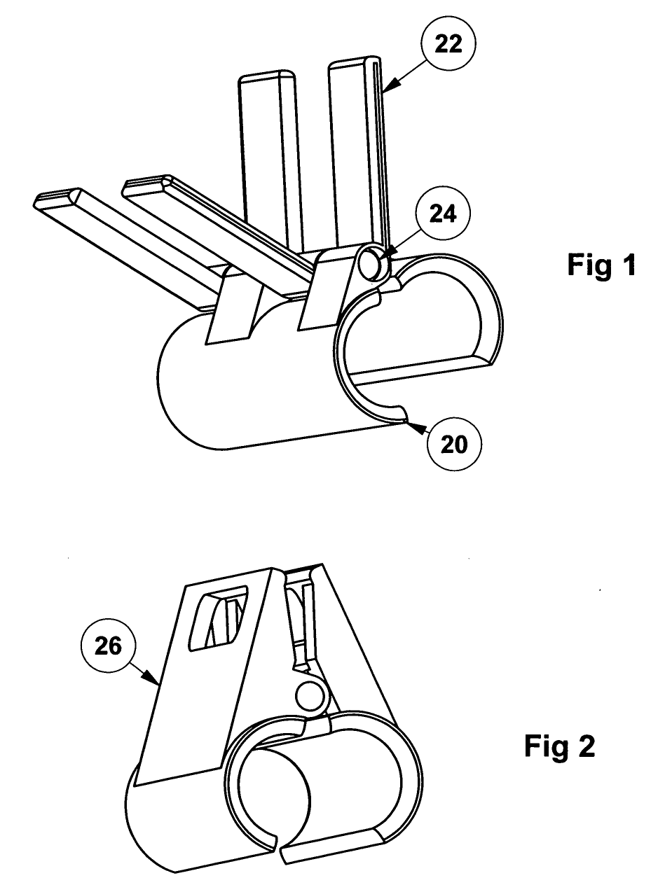

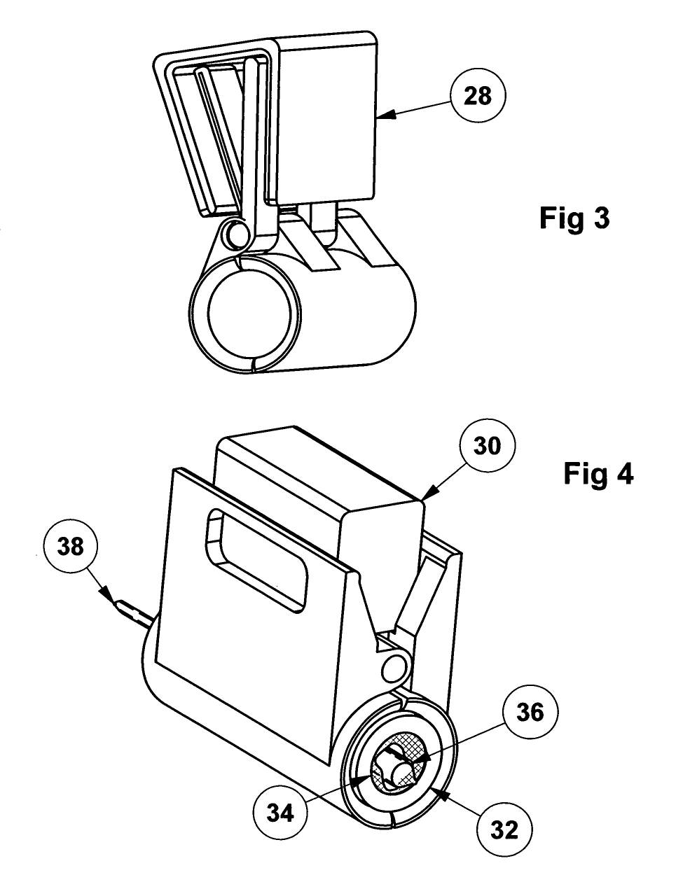

The preferred embodiment to assist balloon angioplasty is the type-A clamp assembly, FIGS. 1 & 3. It consists of an assembly of two opposed action clamshell halves #20. The two halves may or may not be identical in construction. Each clamping clamshell half has an integral extension handle, #22. A pivot pin, #24 unites the two halves into an assembly for pivoted rotary motion. The assembly behaves like a pair of scissors, whereby when the extension handles, #22, of each half are pressed closer, the clamshell halves close to form a duct shaped support for a lumen. Positive U-shaped clamp, #28, can be inserted around the extension handles, #22, to prevent the clamshell halves, #20, from opening, when the balloon is pressurized. The inside radius of the clamshell halves is correctly sized for a given lumen undergoing angioplasty procedure. The clamp assembly is placed to surround the lumen, by reaching its deceased section through an incision in the body. When the angioplasty procedure is completed, the positive stop #28 and clamshell clamp assembly is removed, and the incision sutured shut.

When the balloon is pressurized, the radial forces tending to stretch the lumen outwardly are transmitted into the clamshell's halves, which are in close proximity to the lumen's outside wall. Such wall support facilitates higher pressure inside the balloon, since there is no stretch allowed to burst the lumen. The higher pressures result in more flattening of the plaque, resulting in larger fluid passage created.

### Description and Operation, Alternate Embodiments

First alternative embodiment of the clamshell clamp design is per FIG. 2, where the pivoted movement is opposite of a scissors action. Here separating the extension handles at #26 closes the clamshells. A block used as a wedge, #30 at FIG. 4, is used to prevent the handles from collapsing under the reactionary forces opposing balloon pressure, #36 and #38, on plaque deposits #34 at lumen #32.

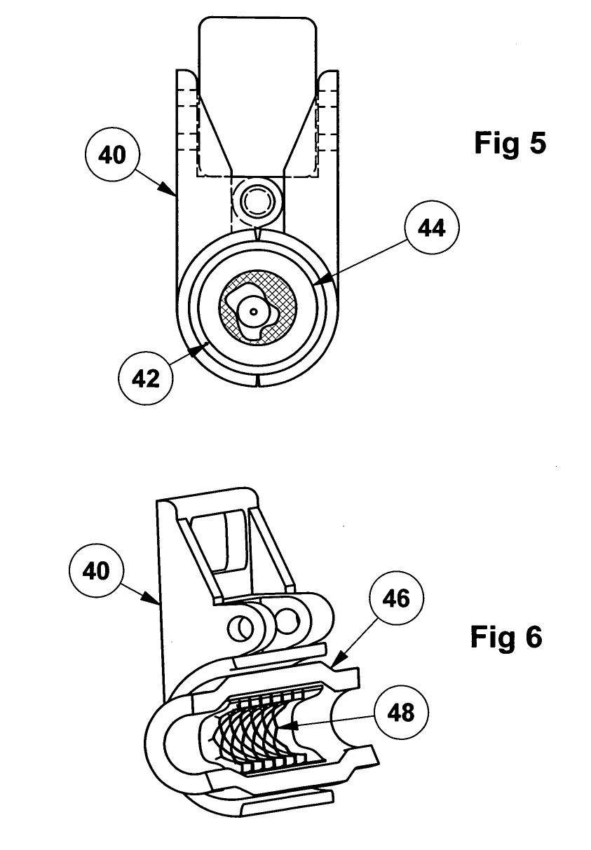

Second alternative embodiment incorporates clamshells whose inside diameter is sufficiently larger than the outer diameter of the lumen, shown at FIG. 5, #40. Radial gap over the lumen wall to clamshell inside radius, before any permanent stretch, is shown as #42, over lumen with plaque #44. This will allow a certain amount of safe level of permanent stretch of the lumen's diameter. The extra stretch into the radial gap allows further increase of the inner diameter of the lumen, aiding in installation of a stronger stent with larger inner diameter. These features are illustrated in the cutaway view of FIG. 6, #46 and #48.

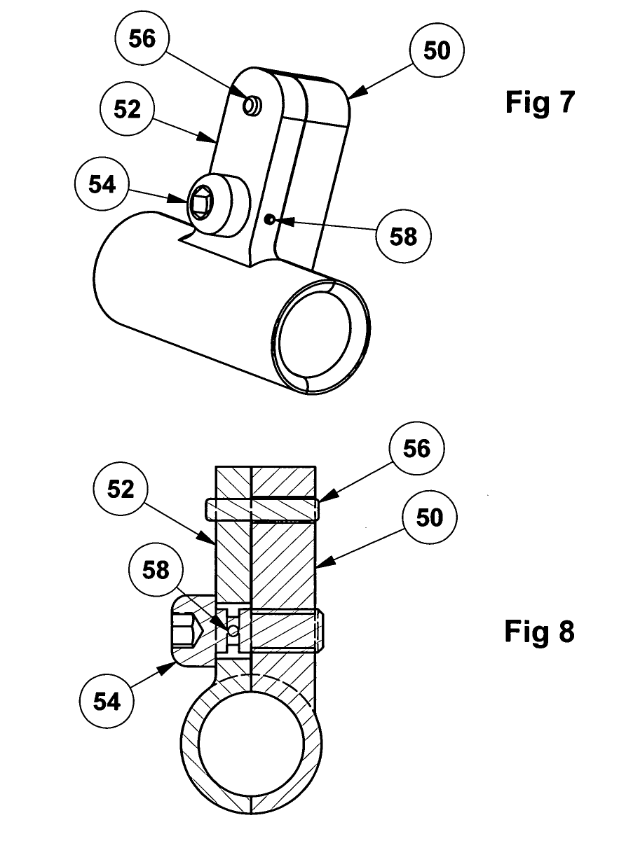

Third alternative embodiment consists of clamshell clamp halves of FIGS. 7 and 8. The clamshell halves, #50 and #52, are easily separable by unscrewing the fastener #54. Yet, the fastener itself is not allowed to be separable from clamshell half #52 by means of one or more pins, #58, radially penetrating into the circumferential groove in the fastener #52. For a different purpose pin #56 is press fitted into clamshell #52. Its function is to align the two clamshell halves, #50 and #52. It, together with the fastener, also prevents any rotational misalignment between the two clamshell halves. It may be readily seen that the clamshell halves may be assembled around a lumen, and subsequently after balloon angioplasty procedure, they can be disassembled for removal from the body.

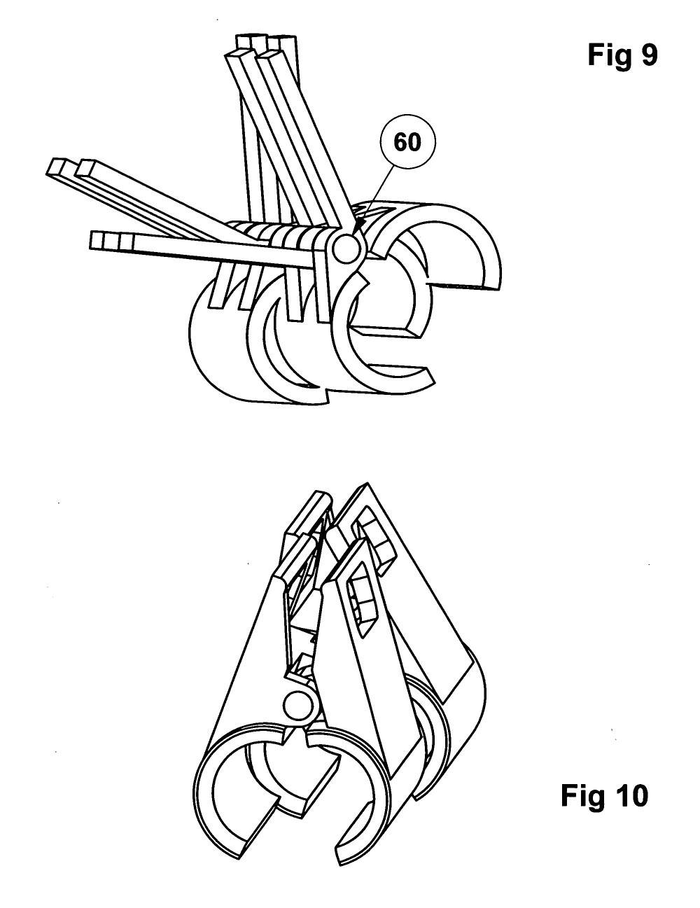

FIG. 9 shows four clamshell halves making up type D clamp assembly. Its individual clamshell halves resemble #20. These are assembled on a common pivot pin #60. Similar to FIG. 9, FIG. 10 illustrates type E clamshell clamp assembly. Its individual clamshell halves resemble #26.

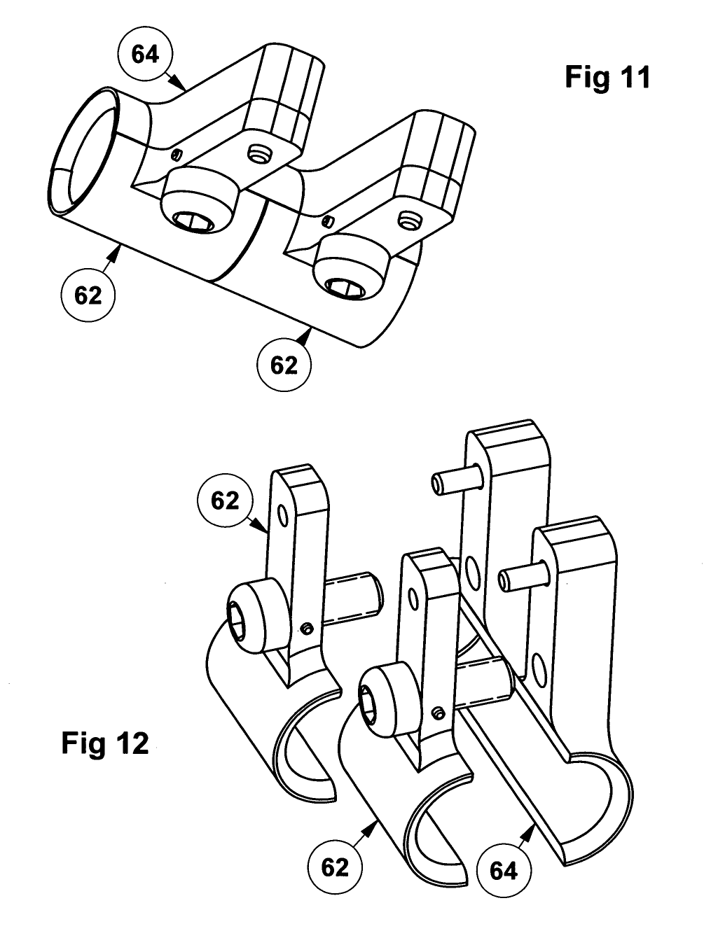

FIGS. 11 and 12 illustrate type F clamp assembly with three clamshell halves. The two clamshell halves #62 carrying fasteners resemble #52; whereas the single clamshell half #64 with threaded holes partially resembles #50.

FIGS. 9 through 12 illustrate clamp assemblies, each constructed with more than two clamshells. All others have each two clamshells, the minimum required.

Conclusions, Ramifications, and Scope:

Accordingly, the reader will see that the invention presented here need not be limited to normal angioplasty with and without stents, internal to the lumen. The invention may be used to install an external stent on a lumen with the internal balloon positioned to act as the force provider. External stent segments can be a part of the clamshell clamp assembly. The compressive forces generated between the inside of clamshell surface and the outside of the lumen may be beneficially used to permanently install external stents. Such external stents, carrying medications, may dispense the medications in a metered fashion to parts of the body external to the lumen. The invention and outlined procedure may be used to install internal and external stents concurrently or successively. The invention may be used during re-catheterization of a lumen already installed with a stent. Since the invention will permit higher balloon pressures, the procedure may be used to attach other types of devices of sturdier design to a lumen, either internally or externally.

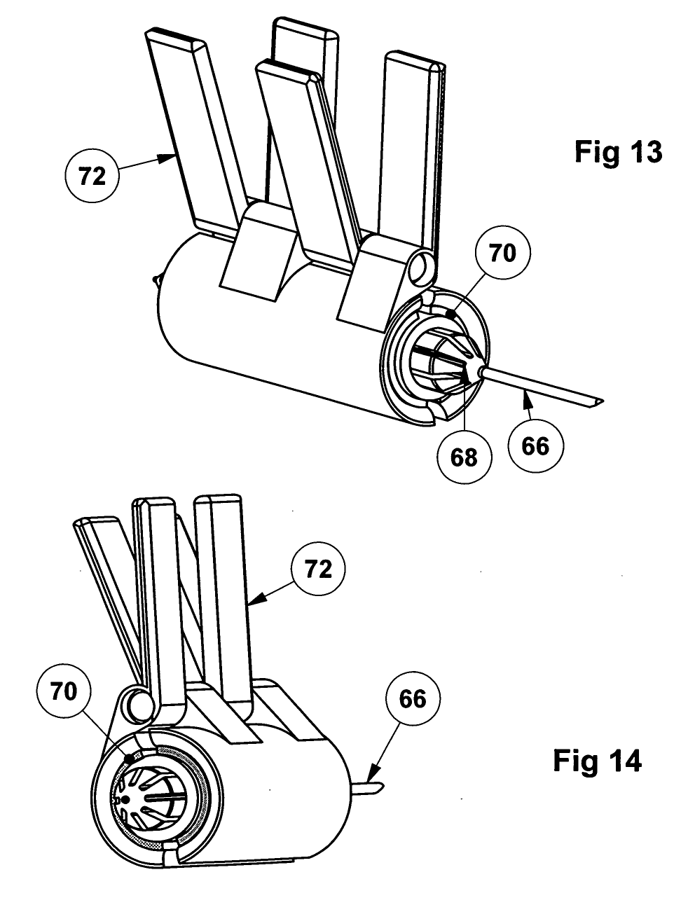

A further possibility is, being able to attach an external device to a lumen using a solid tipped catheter as a reaction-taking anvil, and the clamshells carrying the device providing the clamping force required. Such a possibility is shown in FIGS. 13 and 14. #66 is a catheter, whose solid tip has fluid passages, # 68, of enough area for the brief period of surgery. # 70 is an externally attachable stent or device and #72 is a clamshell assembly through the halves of which clamping force is transmitted. The clamping force may be exerted manually with fingers, or mechanically with pliers-like tool.

I claim:

1. An apparatus for external support of a lumen of a living body during balloon angioplasty procedure, comprising:
A. a plurality of substantially circular arc shaped clamp portions pivotably connected in a manner that, when a fractional number of said clamp portions having identical rotational orientation and thus forming one group, are rotated clockwise, and the remainder of said clamp portions having identical but opposed rotational orientation to said one group and thus forming an opposing group, are rotated counter-clockwise, about said pivot, a substantially closed tubular structure is formed,
B. a means of rotating said clamp potions relative to each other about said pivot,
C. a means of retaining said clamp portions in said tubular form against external forces tending to open said clamp portions,
whereby when said clamp portions in open position are inserted over said lumen and subsequently closed and retained in closed position, they form said tubular structure around said lumen to support said lumen during said procedure.

2. The apparatus of claim 1 wherein:
said means of rotating said clamp portions is substantially a plurality of extension handles beyond said pivot, each of said handles being connected to one of said clamp portions, thereby forming two opposed groups of handles,
whereby when said groups of handles are rotated in opposite directions to come closer to each other, corresponding said groups of clamp portions rotate to come closer to each other to form said closed tubular structure.

3. The apparatus of claim 2 wherein:
said means of retaining said clamp portions in closed position is substantially a u shaped clamp inserted around outside of said extension handles.

4. The apparatus of claim 1 wherein:
said means of rotating said clamp portions is substantially a plurality of extension handles beyond said pivot, each of said handles being connected to one of said clamp portions, thereby forming two opposed groups of handles,
whereby when said groups of handles are rotated in opposite directions so as to move away from each other, corresponding said groups of clamp portions rotate to come closer to each other to form said closed tubular structure.

5. The apparatus of claim 4 wherein:
said means of retaining said clamp portions in closed position is a block inserted between said extension handles,
whereby said block functions as a wedge to prevent opening of said clamshell clamps.

6. An apparatus for external support of a lumen of a living body during balloon angioplasty procedure, comprising:
a plurality of substantially circular arc shaped clamp portions that can be aligned and fastened securely to form substantially a closed tubular structure,
whereby when said clamp portions in unfastened open state are inserted over said lumen and subsequently aligned and securely fastened closed, said clamp portions form said tubular structure around said lumen to support said lumen during said procedure.

7. A catheter to support the inside wall of a living body's lumen comprising a substantially solid cylindrical tip at one end of said catheter with a plurality of fluid passages,
whereby, during a surgical procedure, radial forces may be applied externally on the outside surface of said lumen wall at the location of said tip, without inwardly collapsing said lumen.
